(19) United States
(12) Patent Application Publication
(10) Pub. No.: US 2002/0182609 A1
(43) Pub. Date: Dec. 5, 2002

(54) Microsphere based oligonucleotide ligation assays, kits, and methods of use, including high-throughput genotyping

(75) Inventor: Santosh S. Arcot, Austin, TX (US)

Correspondence Address: PATENT ADMINSTRATOR, KATTEN MUCHIN ZAVIS, SUITE 1600, 525 WEST MONROE STREET, CHICAGO, IL, 60661, US

(73) Assignee: Luminex Corporation

(21) Appl. No.: 09/931,449

(22) Filed: Aug. 16, 2001

Related U.S. Application Data
(60) Provisional application No. 60/225,656, filed on Aug. 16, 2000.

(51) Int. Cl. C12Q 1/68; C07H 21/04

(52) U.S. Cl. 435/6; 536/25.32

(57) ABSTRACT

The present invention teaches a novel approach to detecting and/or analyzing nucleic acid sequences. Generally, the invention relates to detecting and/or analyzing nucleic acid sequences using microsphere-based assays. More specifically, the invention relates to detecting and/or analyzing nucleic acid sequences using microsphere-based oligonucleotide ligation multiplexed assays. The present invention also provides methods and kits for performing microsphere-based oligonucleotide ligation assays, which include bound probes attached to addressable microspheres and free probes bearing a detectable label.

The present invention claims the benefit of priority from U.S. Provisional Application No. 60/225,656, filed Aug. 16, 2000, which is herein incorporated by reference.

### 1. FIELD OF THE INVENTION

The present invention relates to assays for the detection and analysis of nucleic acid sequences. For example, the present invention is useful for the detection of polymorphisms such as single nucleotide polymorphisms (SNPs), and genetic abnormalities such as the cystic fibrosis transmembrane conductance regulator (CFTR) gene mutations.

### 2. BACKGROUND

The recent decoding of the human genome sequence has ushered biology into a new era by generating vast amounts of genome sequence information. Although much remains to be deciphered regarding the identities and numbers of genes and their functional roles in various traits and diseases, significant strides are already being made in cataloging the nucleotide sequence variation among individuals and among disease susceptibility mutations within genes. Altschuler et al., Nature 407: 513-516 (2000).

The analysis of this information in a rapid and cost-effective fashion will undoubtedly require new assays and technologies. A number of technologies and assays such as DHPLC, and real-time PCR-based assays utilizing molecular beacons and the TaqMan system, have been used successfully for genetic analysis. Spiegelman et al., Biotechniques 29: 1084-1090 (2000); Holland et al., Proc. Natl. Acad. Sci. USA 88: 7276-7280 (1991); Tyagi et al., Nat. Biotech. 16: 49-53 (1998). Although these technologies provide a relatively simple assay embodiment, they also suffer from the lack of adequate throughput. The inherent serial nature of these assays (DHPLC) and the difficulty in obtaining numerous fluorescent reporters for high degree of multiplexing (TaqMan and molecular beacons) appear to be some of the factors limiting their throughput.

High throughput is particularly important for multiplex assays using genetic markers such as SNPs for the detection and analysis of complex genetic traits and genetic diseases. Methods and assays that are rapid, economical and amenable to high-throughput embodiments are required. For example, while it is not certain as to how many SNP markers are sufficient to perform whole genome scans, it is becoming apparent that the numbers may range from several tens of thousands to hundreds of thousands of markers.

A typical genotyping assays usually involve several steps; PCR amplification of the target sequence is the first step in most assays, while assays which do not require PCR amplification usually require a relatively large amount of input DNA. One such assay is the Invader Assay (Third Wave), which may require as much as 100 ng of DNA per assay. This requirement, coupled with the inherent difficulty in multiplexing large numbers of markers, indicates that non-PCR based assays may be impractical for large-scale genotyping applications.

Although chip-based genotyping methods can be amenable to high throughput requirements in terms of the number of markers analyzed simultaneously, the high cost of custom synthesis and manufacturing inconsistencies are issues to consider. Mass spectroscopy on chips (Sequenom) is yet another system capable of handling the throughput and cost issues, but one that involves additional post-PCR sample processing steps prior to analysis. Hybridization to high-density oligonucleotide arrays and direct sequencing are also commonly used methods for scoring SNPs. Recently, assays based on oligonucleotide ligation and primer extension or single base chain extension (“SBCE”) have been successfully utilized for high-throughput genotyping applications. Both methods have been used in conjunction with hybridization of the labeled products to oligonucleotide arrays that serve as “tags” or “zipcodes”. These assays usually require a system that is capable of detecting 4 colors simultaneously for the 4 dideoxy nucleoside triphosphates (NTPs) for maximal throughput. In the absence of multi-color detection, a separate reaction has to be performed for each one of the 4 nucleotides, thereby limiting the overall throughput. Other issues include the difficulty of appropriately designing the zipcode primer arrays to prevent self-priming and mispriming of the targets, particularly in a multiplex embodiment. Thus, there is a need in the art for an assay which is accurate, relatively simple, amenable to high-throughput genotyping, and which can be readily optimized.

### 3. SUMMARY

The present invention teaches a novel approach to detecting and/or analyzing nucleic acid sequences. Generally, the invention relates to detecting and/or analyzing nucleic acid sequences using microsphere-based assays. More specifically, the invention relates to detecting and/or analyzing nucleic acid sequences using microsphere-based oligonucleotide ligation multiplexed assays. Preferably, the present invention is adapted for use with spectrally addressable microspheres, such as LabMAP™ available from Luminex Corp., and flow analyzers, such as the Luminex 100 analyzer, capable of sampling all 100 subsets of spectrally addressable microspheres simultaneously. Use of spectrally addressable microspheres with flow analyzers capable of resolving the different subsets, can currently enable simultaneous sampling of up to 100 targets, can provide fluorescent reporter intensity values without the need for additional sample processing, and thus can provide rapid, economical, and/or high throughput as compared to other currently available methods of detecting and/or analyzing nucleic acid sequences.

In one aspect, the present invention relates to a method of detecting and/or analyzing nucleic acid sequences, comprising: (a) contacting a sample suspected of containing at least one target nucleic acid sequence with at least one subset of free probes and at least one subset of spectrally-addressable bound probes; (b) allowing the at least one subset of free probes and the at least one subset of spectrally-addressable bound probes to hybridize to the target nucleic acid sequence, if present; (c) ligating the free probes hybridized to the target nucleic acid with the bound probes hybridized to the target nucleic acid to provide ligated products; and (d) detecting the presence of the ligated products. In some embodiments, method further comprises: contacting the sample with polymerase chain reaction (PCR) reaction components, including effective amounts of thermostable DNA polymerase, deoxy nucleotide triphosphates (dNTPs), and PCR primers complementary to sequences upstream and downstream from the sequence of interest in the target nucleic acid sequence; and amplifying a sequence of interest of the target nucleic acid sequence.

In one aspect, the present invention relates to a method of detecting and/or analyzing nucleic acid sequences, comprising: (a) contacting a sample suspected of containing at least two target nucleic acid sequences with at least one subset of free probes and at least two subsets of spectrally-addressable bound probes; (b) allowing the at least one subset of free probes and the at least two subsets of spectrally-addressable bound probes to hybridize to the at least two target nucleic acid sequences, if present; and ligating the free probes hybridized to the target nucleic acid with the bound probes hybridized to the target nucleic acid to provide ligated products; and (c) detecting the presence of the ligated products. In some embodiments, the free probes have substantially identical nucleotide sequences and detectable labels. In some embodiments, a bound probe comprises a microsphere coupled to an oligonucleotide probe, and each subset of bound probes is distinguishable from another subset based at least on its spectral address and the sequence of its oligonucleotide probe. In some embodiments, a bound probe comprises a oligonucleotide probe coupled to a microsphere, the oligonucleotide probes in a first subset differs from those of other subsets in that the nucleotide(s) found at the free end of the nucleotide probe of the first subset differs from the nucleotide(s) found at the free ends of the nucleotide probes of the other subsets, though the nucleotide sequences of the probes of each of the subsets can otherwise be substantially identical.

In one aspect, the present invention relates to a method of detecting and/or analyzing nucleic acid sequences, comprising: (a) contacting a sample suspected of containing at least two target nucleic acid sequences with at least two subsets of free probes and at least one subset of spectrally-addressable bound probes; (b) allowing the at least two subsets of free probes and the at least one subset of spectrally-addressable bound probes to hybridize to the at least two nucleic acid sequences, if present; and ligating the free probes hybridized to the target nucleic acid with the bound probes hybridized to the target nucleic acid to provide ligated products; and (c) detecting the presence of the ligated products. In some embodiments, the method comprises contacting a sample suspected of containing at least two target nucleic acid sequences with at least one subset of free probes and at least two subsets of bound probes. In some embodiments, the bound probes comprise a nucleotide probe coupled to a microsphere, the microsphere-bound probes have substantially identical nucleotide sequences. In some embodiments, a free probe comprises a nucleotide sequence and a detectable label, the free probes of one subset are distinguishable from the free probes of another subset based at least on the nucleotide sequence. In some embodiments, a free probe comprises a nucleotide sequence and a detectable label, the detectable label and the portion of the nucleotide sequence found at the end of one subset of probes differs from the detectable label and the portion of the nucleotide sequence found at the end of another subset of probes, though the nucleotide sequences of the sets of free probes can be otherwise substantially identical.

In one aspect, the present invention relates to a method of detecting and/or analyzing nucleic acid sequences, comprising: (a) contacting a sample suspected of containing at least two target nucleic acid sequences with at least two subsets of free probes and at least two subsets of spectrally-addressable bound probes; (b) allowing the at least two subsets of free probes and the at least two subsets of spectrally-addressable bound probes to hybridize to the target nucleic acid sequence, if present; and ligating the free probes hybridized to the target nucleic acid with the bound probes hybridized to the target nucleic acid to provide ligated products; and (c) detecting the presence of the ligated products.

The present invention also provides a kit for performing a microsphere-based oligonucleotide ligation assay, which comprises bound probes attached to spectrally addressable microspheres and free probes bearing a detectable label. Preferred kits of the invention contain at least two subsets of microspheres or at least two sets of free probes. The kits may further include a thermostable ligase, one or more reagents for effecting nucleic acid amplification, and one or more reaction buffers.

Specific embodiments of the present invention may be directed to one, some or all of the above- or below-indicated aspects as well as other aspects, and may encompass one, some or all of the above- or below-indicated embodiments as well as other embodiments. Such other embodiments and applications of the present invention will become apparent to those of ordinary skill in the art after consideration of the present disclosure.

### 4. BRIEF DESCRIPTION OF THE DRAWINGS

FIG. 1 is a schematic diagram of two of the exemplary embodiments that can be utilized for a microsphere based OLA.

FIG. 2 is a pair of charts showing the effect of varying the input amount of PCR amplified target DNA on the OLA reaction.

FIG. 3. is a pair of charts showing the effect of varying the input amount of biotinylated free probe on the OLA reaction.

FIG. 4 is a pair of charts showing the effect of varying the amount of thermostable ligase on the OLA reaction.

FIG. 5 is a pair of charts showing the effect of varying the number of cycles in a thermal-cycler on the OLA reaction.

FIG. 6 is a schematic diagram of an embodiment which combines the steps of PCR amplification of the target sequence and the OLA reaction in a single reaction vessel.

### 5. DETAILED DESCRIPTION

5.1 Definitions

The term “detecting” is understood to mean identifying the presence or absence of a nucleic acid sequence.

The term “analyzing” is understood to mean determining or confirming the sequence of a nucleic acid sequence.

The term “target” is understood to mean any substance desired to be detected or analyzed, which is suspected of being in the sample to be analyzed. Thus, “target nucleic acid sequence” is understood to mean any nucleic acid sequence desired to be detected or analyzed and suspected of being in a sample. The target nucleic acid sequence may be only a portion of a larger sequence in the sample. The samples to be assayed, therefore, may contain nucleic acid molecules with one or more known or suspected sequences, for example, polymorphic sequences such as single nucleotide polymorphisms, deletions, or other genetic variations or mutations. The terms “oligonucleotide molecule” and “polynucleotide molecule” are understood to mean linear DNA or RNA molecules having a 3′ end and a 5′ end, and a known, partially known, or predetermined nucleic acid sequence, but have been used interchangeably herein. The terms “oligonucleotide molecule” and “polynucleotide molecule” are also sometimes shorthanded herein as “nucleic acid molecule” or “target molecule.” The “oligonucleotide molecule” or “polynucleotide molecule” or only a portion thereof may be the “target nucleic acid sequence.” The term “known,” when used with regard to a nucleic acid molecule, is understood to refer to a nucleic acid molecule whose sequence has been previously identified.

The term “predetermined,” when used with regard to a nucleic and molecule, is understood to refer to a nucleic acid molecule whose sequence has been artificially derived and is thus known. The term “probe” is understood to mean a molecule that can bind to at least a portion of a target nucleic acid sequence. The probe molecule is typically a nucleic acid sequence that is substantially complementary to at least a portion of the target nucleic acid sequence. The probes or a given subset have substantially identical nucleotide sequences.

The term “substantially complementary” when used in connection with the phrase nucleic acid sequence is understood to mean that one or more nucleotides of the probe differ from what otherwise would be expected, based on the target nucleic acid's sequence, but the probe can nonetheless substantially hybridize to the correct position on the target molecule, i.e. to the target nucleic acid sequence, as appropriate for example, in the OLA reaction.

The term “substantially identical” when used in connection with the phrase nucleotide sequence is understood to mean that one or more nucleotides at one or more positions of probes in a subset may differ due to one or more substitutions, insertions, deletions, or combinations thereof, but can still be distinguished from probes belonging to another subset and can substantially hybridize to the correct position on the target molecule, i.e. to the target nucleic acid sequence, as appropriate for example in the OLA reaction.

The term “free probe” is understood to mean a probe, which is not bound to a solid support, and which may be labeled for detection. “Free probes” can be oligonucleotides which have a detectable label attached at one end, the oligonucleotide sequence of the probe being substantially complementary to portions of the target molecule's sequence.

The term “bound probe” is understood to mean a probe which is bound to a solid support, and in which the solid support is labeled for detection. Optionally, the probe can also be labeled. The solid support is typically a “particle” suitable for use in flow analyzers, preferably multiplex flow analyzer assays. One skilled in the art will recognize that one type of “particle” may be man-made beads or microspheres. Microspheres or beads are generally known in the art and may be obtained from manufacturers such as Sperotech, Bangs Laboratories, or Polymer Labs. The terms bead, microsphere and particle are used interchangeably hereinafter. Preferably, the solid support is labeled by incorporating fluorochromes within the solid support. For example, but without limitation, the invention may be practiced using the LabMAP™ platform (available from Luminex, Austin, Tex.) of spectrally addressable microspheres and flow analyzers. This platform includes subsets of microspheres which separately contain one or more fluorophores or concentrations of fluorophores which can be detected and analyzed using a flow analyzer capable of distinguishing between the frequencies, intensities, or refractive indices of scattered light during detection, e.g. see Chandler et al., U.S. Pat. No. 5,981,180. Preferably, the “bound probe” is attached at one end to a spectrally addressable bead or microsphere, most typically coupled via a modifier moiety.

The term “free end” refers to the end of the free or bound probe, which is not attached to the detectable label or microsphere. If the “free end” is at the 5′ end of the sequence, or oligonucleotide, it preferably contains a phosphate modification, for effective use of currently available thermostable ligases. Upon hybridizing to the target molecules, the two free ends of the probes are adjacent to one another and thus the two ends are capable of being ligated by the thermostable ligase to form a microsphere-bound ligation product.

The term “attached” is understood to mean that the items referred to are bonded together by either covalent, ionic, or other chemical bonds, or coupled together via a modifier moiety. Examples of modifier moieties include, but are not limited to, those that introduce a primary amine to the 5′ or 3′ end of an oligonucleotide to permit a carbodiimide coupling of the oligonucleotide to carboxylic acid group on the microsphere's surface. However, it is contemplated this attachment can be accomplished by using other biomolecular coupling chemistries, such as amino-hydroxyl, hydrazide, amide, chloromethyl, aldeyde, or tosyl moieties. The term “subset of free probes” and “subset of bound probes” refers to a group of free or bound probes sharing essentially the same characteristics. By “essentially” it is meant that the free or bound probes are similar to the extent that they can be identified as belonging to the same subset of free or bound probes and also distinguished from the other subsets of free or bound probes. The term “spectrally-addressable” is understood to mean labeled in a distinguishable manner. More specifically, a subset of spectrally-addressable bound probes has a unique label that distinguishes that subset from other subsets. Preferably, the label is one or more fluorochromes incorporated within the particle, imparting a unique flourescence emission spectrum to the particles of a subset. By “unique” it is meant that the fluorescence emission spectrum of particles in one subset are distinguishable from the fluorescence emission spectrum of particles of another subset. The term “spectral address” used in connection with bound particle therefore is understood to mean the unique flourescence emission spectrum of the bound particle.

The term “sequence” is understood to mean an ordered arrangement of one or more nucleotides joined to form a sugar-phosphodiester backbone, as in a DNA or RNA sequence of nucleotides, and is typically denoted by a single letter identifying the nitrogenous base (guanosine G, cytosine C, thyminosine T, adenosine, A, urcine U) attached thereon.

The term oligonucleotide ligation reaction refers to the enzymatic reaction joining adjacent ends of nucleic acid molecules that are hybridized to a template or target molecule, e.g. see Nikiforov, U.S. Pat. No. 5,952,174.

5.2 Description

In the following detailed description, reference is made to the accompanying drawings and individual exemplary assays. These drawings and exemplary assays are shown and described by way of illustration of specific embodiments in which the invention may be practiced. It is to be understood that other embodiments may be utilized without departing from the scope of the present invention.

The exemplary microsphere-based oligonucleotide ligation assays provided herein include several steps. These assays include allowing the free probes and the bound probes to hybridize to the target nucleic acid molecules, if present; ligating the free ends of the bound probes together, to provide microsphere-bound ligated products; and detecting the presence of microsphere-bound ligated products. In the description which follows, examples may indicate that free end of the free probe is the 5′ end and the free end of the bound probe is the 3′ end. However, a person of ordinary skill in the art will appreciate that for many embodiments, the invention can be modified so that the free end of the of the free probe is the 3′ end, rather than the 5′ end, and the free end of the bound probe is the 5′ end, rather than the 3′ end. Such modifications are within the scope of the present invention. Thus, generally, the free end of the bound probe should be opposite that of the free probe (so the sequences of the free and bound probe may be ligated together) and it is not essential that the free probe have the 5′ free end and the bound probe have the 3′ free end.

Ligation is accomplished by any viable means, typically and most conveniently, using a thermostable ligase, examples of which are well known in the art and several species of which are commercially available, e.g., Taq Ligase (New England Biolab), or Pfu Ligase (Stratagene, La Jolla).

The assays of the present invention may be carried out, if desired, in separate reaction vessels, at least one for each set of free probes, particularly if only one detectable label is used for multiple sets of free probes. Preferably, the assays are carried out in a single reaction vessel. Advantageously, the detectable label may be any moiety that is capable of being detected, either directly or through the action of an intermediate step or substance. For example, a detectable label may comprise a fluorescent dye, a radiolabel, a spectrally addressable microsphere, or one member (e.g., biotin) of a pair of proteins (e.g., biotin-streptavidin) exhibiting a strong binding affinity for one another. The other member of the pair may be conjugated to a label, including but not limited to, a radiolabel, a fluorescent label, a bioluminescent label, a chemiluminescent label, a nucleic acid label, a hapten label, an enzyme label, and the like.

Hence, a method is herein described that allows for rapid and economical high-throughput genotyping in an automatable fashion with a minimal number of steps.

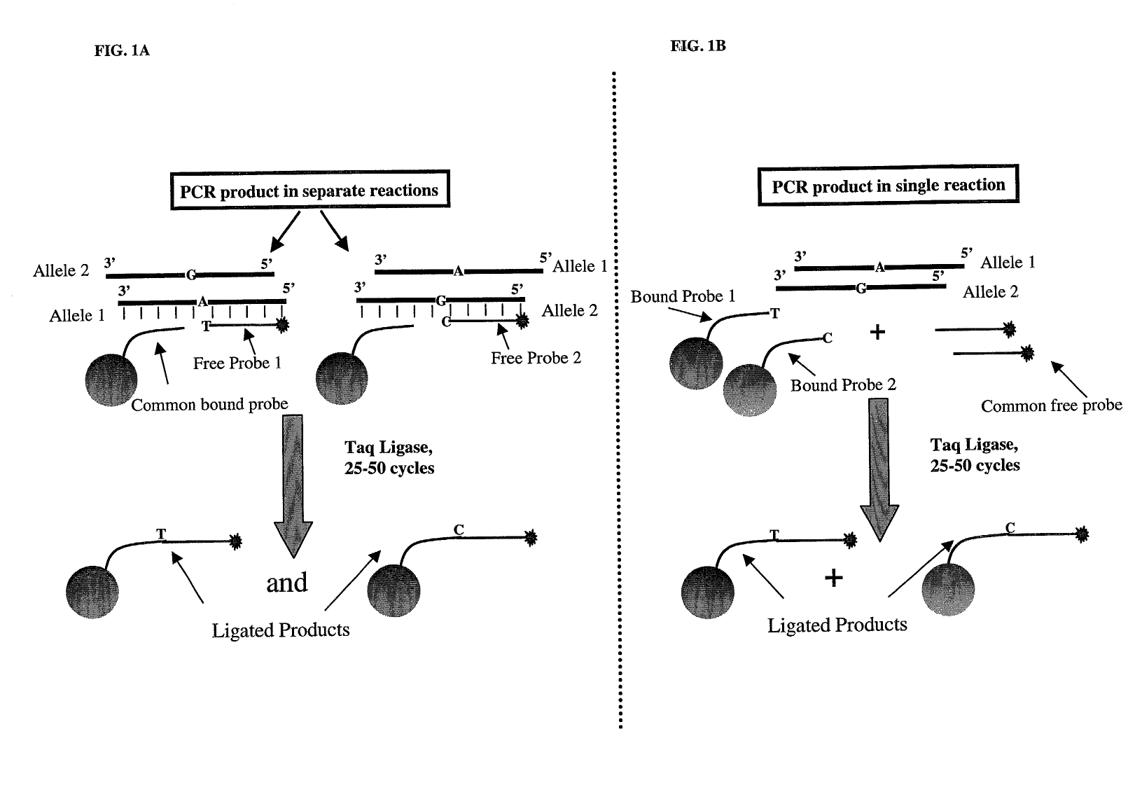

FIG. 1 depicts a schematic diagram of two of the embodiments using OLA coupled to spectrally addressable microsphere technology. The PCR amplified target used in this example represents a target that is heterozygous for an SNP (shown as Allele 1 & 2). Embodiment 1 is depicted in FIG. 1A. In FIG. 1A, the probe that is coupled to the microspheres via its 5′ end is common to both alleles, and the 3′ end of this probe stops short of the polymorphic nucleotide. The two free probes (shown as Free Probe 1 & 2) have the appropriate bases complementary to the target's polymorphic bases at their 5′ ends and biotin modification at their 3′ ends (shown as stars). In addition, the 5′ ends of the free probes are phosphorylated, as required for an enzymatic ligation reaction. The biotin molecule at the 3′ end serves as a reporter for monitoring the success of the ligation reaction. Also, since a single reporter or detectable label is used for detection, the free probes should be used in separate reactions. The separation of the reactions can be accomplished by placing the amplified DNA and OLA reactants into separate tubes. The bound probe is designed to anneal just proximate to the polymorphic nucleotide, and is common for both alleles. Here, the free probes serve a dual function of discrimination and detection. Since the most convenient forms of detection may not involve the use of two distinct labels, the two genotyping probes are preferably used in separate reaction vessels. However, it is also recognized that by selecting distinguishable detectable labels for each set of free probes, this embodiment may also be performed in a single reaction vessel.

Embodiment 2 is depicted in FIG. 1B. In FIG. 1B, the two bound probes have bases which are complementary to the target molecule's polymorphic bases at their respective 3′ ends, and are coupled to two spectrally distinct subsets of microspheres. In this embodiment, the free probe is common to both alleles, is phosphorylated at the 5′ end, and has a biotin reporter on the 3′ end (depicted as stars). In both embodiments, a successful ligation reaction results in a product that can be detected via the biotin reporter. In Embodiment 1, the OLA reaction for the SNP pair and the subsequent detection of a successful reaction is performed in separate reactions since the microsphere set used for discrimination of the alleles is identical. In Embodiment 2, since the probes for the 2 alleles are individually coupled to spectrally distinguishable microsphere sets, the reaction and detection can be performed in a single tube. For both embodiments, the targets comprised PCR amplified product from DNA samples that were heterozygous and homozygous for an SNP (shown as “heterozygote” and “homozygote”).

In either of these embodiments, a typical assay kit may have the following reaction components: PCR amplified target molecules and/or PCR reactants, probes coupled to beads, free probes (i.e. detection probes), reaction buffer and thermophilic ligase. All the components are added into a single reaction mixture and subjected to repetitive two-cycle denaturation and ligation using a thermal cycler for 25-40 cycles or as appropriate for the given probes and target molecules. Separate reaction vessels may also be used when appropriate, per Embodiment 1. At the end of the reaction, unused free probes are preferably (though not necessarily) separated from the microspheres, such as by filtration or centrifugation. Detection of the ligated products is accomplished, for example, by incubation with streptavidin conjugated to phycoerythrin (SAPE) using a flow analyzer, such as the Luminex 100 analyzer or a conventional flow analyzer.

The invention is further illustrated as follows.

### 6. Example 1

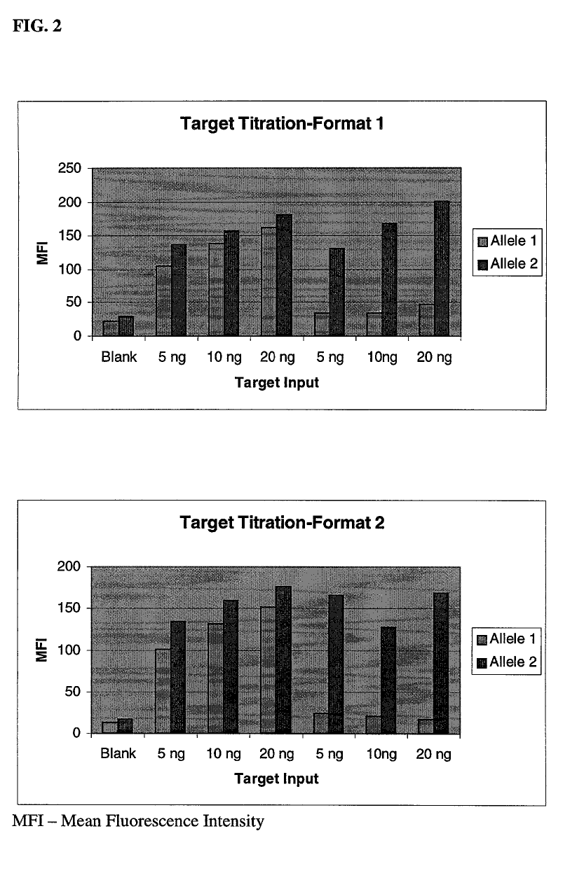

In this example, two different genomic DNA samples were used to generate 242-bp PCR amplicons from HLA-DQA1 loci that harbored a G/C SNP. One sample was heterozygous for the SNP while the other was homozygous for the C allele. Using these two samples, both Embodiments were analyzed for factors that affect the efficiency of the microsphere-based OLA genotyping. Optimization results are shown in FIG. 2 to FIG. 5, and described below.

6.1 PCR Amplification of Targets

Targets for OLA comprising 242 bp fragment from the HLA class II DQA1 locus. PCR amplification was performed using primers DQA-A (5′GTGGTGTAAACTTGTACCAGT 3′) and DQA-B (5′TTGGTAGCAGCGGTAGAGTTG3′). Typical amplification reactions included I micromolar of each primer, 200 micromolar of each nucleotide (dNTPs), reaction buffer (Quiagen, Valencia, Calif.), Thermostable Polymerase (2.5 units) (Quiagen, Valencia, Calif.) and 100 ng genomic DNA as template in a 50 microliter reaction. Each reaction was subjected to the following amplification cycle: 30 sec at 94 C. (denaturation), 45 sec at 60 C. (annealing), and 45 sec at 72 C. (extension). This process was repeated for 20-50 cycles using a PE9700 thermal cycler.

6.2 Target Quantification

PCR amplified material (5 microliters) was fractionated on an agarose gel and stained with ethidium bromide to visualize successful amplification. Approximate quantification of the correct PCR amplified DNA fragment was accomplished by visual comparison to the various fragments of known DNA mass (DNA Mass Ladder, Life Technologies Inc.) loaded simultaneously on the gel.

6.3 OLA Optimization

The effect of various factors that influence the efficiency of microsphere-based OLA were experimentally measured to identify standard conditions for the OLA reaction. These were determined to be about 10 ng of a 242 bp PCR product, 5000 of each microsphere set with probes coupled to their surface, 50 nM biotinylated free probe and 5 units of Taq DNA Ligase in a 20 μl reaction volume.

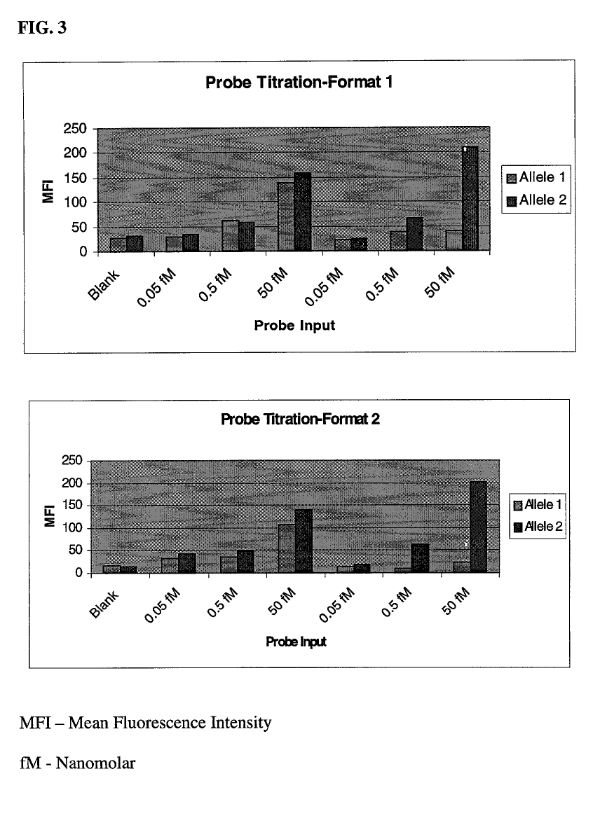

FIG. 2 shows the effect of varying the input amount of PCR amplified DNA target on OLA. The samples marked as blank indicate no DNA as template. The first 3 sets represent results of OLA reaction using heterozygous DNA target while the next 3 represent results using DNA target that is homozygous for allele 2. Alleles 1 and 2 represent the two nucleotides that represent this particular single nucleotide polymorphism (SNP), all other nucleotide sequences being identical. FIG. 3 is a pair of charts showing the effect of varying the input amount of biotinylated free probe on the OLA reaction. The samples marked as blank indicate that no DNA template was present in the sample. The first 3 sets represent results of OLA reaction using heterozygous DNA target while the next 3 represent results using DNA target that is homozygous for allele 2. In this example, a 50 nanomolar free probe concentration seems to be an optimal amount for both embodiments.

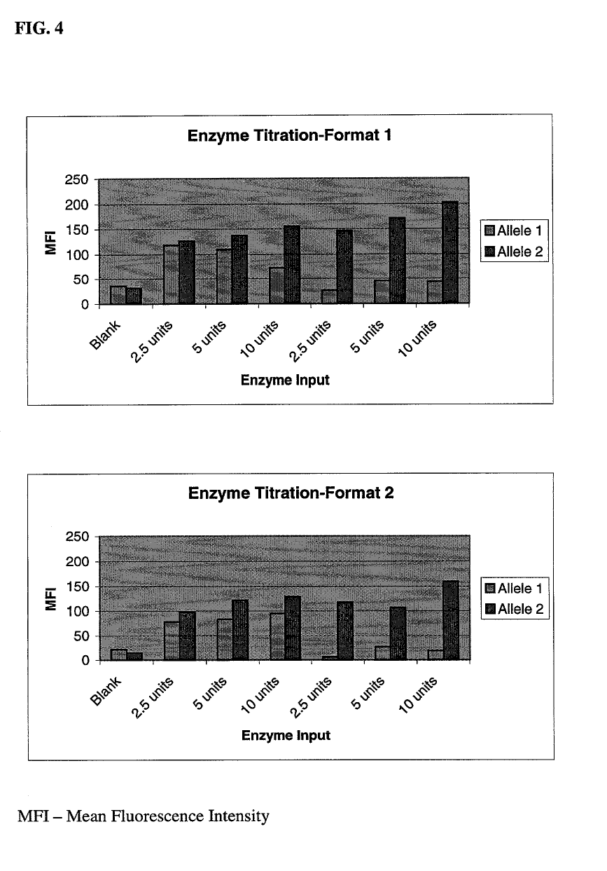

FIG. 4 is a pair of charts showing the effect of varying the amount of thermostable ligase on the OLA reaction. The samples marked as blank indicate no DNA as template. The first 3 sets represent results of OLA reaction using heterozygous DNA target while the next 3 represent results using DNA target that is homozygous for allele 2. As little as 2.5 units of enzyme is sufficient for a successful assay.

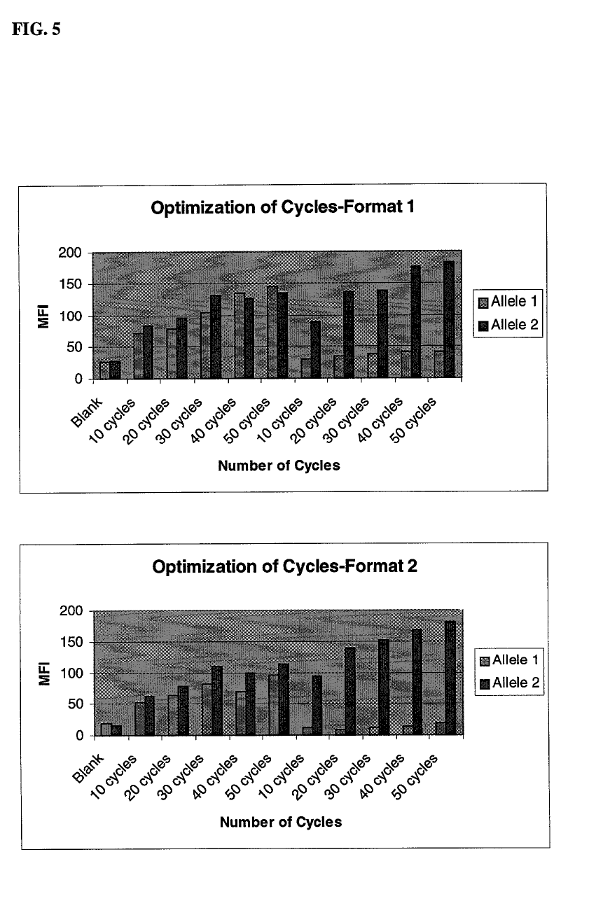

FIG. 5 is a pair of charts showing the effect of varying the number of cycles in a thermal-cycler on the OLA reaction. The samples marked as blank indicate no DNA as template. The first 3 sets represent results of OLA reaction using heterozygous DNA target while the next 3 represent results using DNA target that is homozygous for allele 2. The results show that as few as 10 cycles are sufficient for a distinct signal.

All oligonucleotide probes were synthesized such that their melting temperatures are approximately 55 C.

6.4 Embodiment 1

In this embodiment, the bound probe is common for both alleles. Oligonucleotide sequences for the free and bound probes corresponding to the HLA-DQA1 locus markers 3401 and 3402 were as follows:

DQ340X Bound-5′UnilinkATGAATTTGATGGAGATGAG-3′
DQ3401 Free-5′-pGAGTTCTACGTGGACCTGGA-3′Biotin
DQ3402 Free-5′-pCAGTTCTACGTGGACCTGGA-3′Biotin

The “X” in DQ34OX indicates that the bound probe was common to both alleles. Note, all of the bound probes were synthesized by Operon, Inc. (Alameda, Calif.) with a 5′UniLink™ (Clontech, Palo Alto, Calif.) amino modification. The amino modified probes were then coupled to 5.5 micron carboxylated latex microspheres via a standard one-step reaction. Dunbar et al., Clin. Chem 46:1498-1500 (2000). Coupled microspheres were then stored until use in 10 mM Tris-EDTA, pH 8.0 solution at a concentration of 50,000 microspheres per microliter.

The two free probes corresponding to the 2 alleles in question serve as reporters, and distinguish between Allele 1 and Allele 2. Here, “p” indicates a phosphate modification, while biotin was used in the detection reaction. Note, the 5′ phosphate modification is a requirement for the enzymatic ligation reaction employed.

In this embodiment, 2 individual OLA reaction mixtures were prepared, one with each of the free probes, since for convenience only a single reporter channel was used. The components of the reaction mixture were as follows: 5000 microspheres coupled with bound probes (each bead has approximately 10 e5-10 e6 probes coupled to it), 5-20 ng of PCR amplified DNA as template, 50 nanomolar free probe, 1× reaction buffer (supplied by the vendor) and Taq Ligase (5 to 10 units). (New England Biolabs, Beverly, Mass.). All reagents were present in a total reaction volume of 20 microliters.

6.5 Embodiment 2

In this embodiment, the two allele-specific probes were coupled to 2 spectrally distinct bead sets and had the following sequences:

DQ3401 Bound-5′UnilinkATGAATTTGATGGAGATGAGG-3′
DQ3402 Bound-5′UnilinkATGAATTTGATGGAGATGAGC-3′
DQ340X Free-5′-pAGTTCTACGTGGACCTGGA-3′Biotin

Here, the free probe, DQ340X, was common to both alleles, while DQ3401 corresponds to Allele 1, and DQ3402 corresponds to Allele 2. P also indicates a phosphate modification. In this embodiment, all the reagents for the discrimination between the two alleles were placed in a single reaction vessel since the allele-specific probes were coupled to spectrally distinguishable beads. The components of the reaction mixture were as follows: 5000 beads coupled to each one of the two allele-specific probes (total 10000 beads), reaction buffer (supplied by vendor), 5-20 ng of PCR amplified DNA as template, Thermostable Ligase (5-10 units) and 200 nanomolar free probe. All reagents are present in a total reaction volume of 20 microliters.

6.6 Thermal-cycler Profile for OLA Reaction

The reactions in both embodiment were subjected to the following ligation profile: 1) 92 C. for 4 min; 2) 55 C. for 3 min; 3) 92 C. for 15 sec; and 4) 55 C. for 30 sec. Steps 3-4 were repeated for 25-50 cycles.

6.7 Detection of Ligated Products

Completed OLA reactions were purified via standard means, e.g. filtration or centrifugation, to eliminate excess free probes. The beads were then incubated with a solution containing 100 ng of streptavidin conjugated to phycoerythrin (SAPE, Molecular Probes, Eugene, OR) in a volume of 100 microliters for 10 min and analyzed using a flow analyzer (Luminexl00).

6.8 Interpretation of Results

In Embodiment 1, the two allele-specific probes are assayed in different reactions. A positive signal from both reactions would indicate that the DNA sample being tested is heterozygous. A positive signal from either one of the two reactions alone would indicate that the sample is homozygous for one of the 2 alleles.

In Embodiment 2, a positive signal from both bead sets would indicate the presence of a heterozygous sample while a positive signal from either one of the 2 bead sets would indicate the presence of homozygous sample. Both embodiments allow for multiplexing the assays such that different sequences can be detected and typed in a single reaction, given the availability of a large number of spectrally distinguishable bead sets.

When analyzed using the LabMAP™ system and Luminex 100 analyzer, as the microspheres are drawn into the analyzer, they traverse through the path of two laser beams, a 532 nm green YAG laser and a 635 nm red diode laser. The microspheres in the LabMAP™ system are impregnated with varying proportions of different fluorochromes. The green laser illuminates each microsphere and quantifies the fluorescence intensity resulting from the phycoerythrin reporter on the surface. Similarly, the red diode laser illuminates each microsphere and quantifies the proportion of the internal red and infrared fluorochromes. On the basis of the resulting signal, each microsphere gets classified into one of 100 spectrally distinguishable subsets of microspheres that may be present in the mixture. Electronic gating utilizing the side-scatter property of each microsphere eliminates aggregates and ensures that only individual microspheres are analyzed. A 12 bit digital-to-analog high speed processor converts the reporter fluorescence signal associated with each microsphere and presents the reporter intensity values numerically in real time after subtracting the background fluorescence of the solution. Although thousands of beads pass through the analyzer, at least 100 microspheres from each subset are analyzed and the results presented as the median fluorescence intensity (MFI) value associated with each microsphere subset.

Genotype determination for the bi-allelic marker was accomplished post-hoc by subtracting the MFI values of the negative sample and then determining the ratio of the MFI values for each allele individually against the sum of the MFI values for both alleles. A ratio of between about 0.3 and 0.7 for both alleles indicated a heterozygous genotype, while a ratio greater than 0.8 for one allele and less than 0.2 for the other allele indicated the presence of a homozygous genotype. This relationship was determined empirically over repeated experiments analyzing samples with known genotypes using the HLA-DQA1 markers and, in Example 3, the cystic fibrosis transmembrane (CFTR) markers.

Although the LabMAP™ system was advantageously used herein, it is to be understood that other systems which use different fluorochromes and laser light sources, or which use different identifiable characteristics (e.g., bead size) may be used without departing from the scope of the invention.

### 7. EXAMPLE 2

In this example, the method was also performed on the HLA-DQA1 G/C SNP, but here the steps of PCR amplification and microsphere-based oligonucleotide ligation were combining in a single reaction vessel. PCR amplification of the target sequence in the presence of microspheres had not been demonstrated previously. Likewise, PCR amplification of targets in the presence of thermostable ligase and mixtures of microsphere-bound and reporter-bound oligonucleotide probes had also not been demonstrated. The ability to perform PCR amplification successfully in the presence of subsets of microsphere-bound oligonucleotide probes and subsequently performing a thermostable ligase-dependent genotyping of the amplified product using OLA, all in a single reaction, thus presented a novel problem.

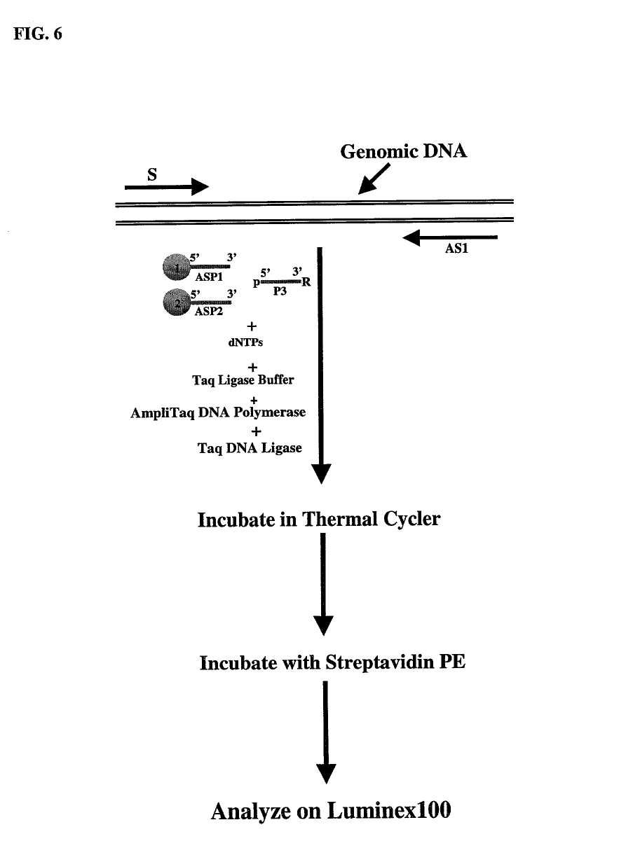

The details of the reaction and assay are shown schematically in FIG. 6. The reaction components include: the genomic DNA sample; PCR primers (S1 and AS1); microsphere sets 1 and 2 with allele-specific oligonucleotide probes ASP1 and ASP2 attached to their surfaces, respectively; oligonucleotide probe P3 containing a reporter molecule R1; deoxynucleotide triphosphate mix (dNTPs); DNA polymerase/ligase reaction buffer; thermostable DNA polymerase; and thermostable DNA ligase. PCR primers S1 and AS1 were designed to have a Tm between 70 C. and 75 C. and were generally 35-40 nucleotides long. Microsphere sets 1 and 2 each have a unique spectral signature by virtue of varying proportions of 2 fluorescent dyes impregnated into them. Oligonucleotide probes ASP1 and ASP2 were attached to the microspheres via an amino-modification at their 5′ ends and had a Tm of approximately 55 C. In this example, ASP1 and ASP2 were identical in sequence except for the base at their 3′ end, which was complementary to the bi-allelic SNP. Oligonucleotide probe P3 also had a Tm of approximately 55 C. and a phosphate at its 5′ end and a biotin molecule (R1) at its 3′ end. In the presence of a DNA target with a sequence that is complementary to that of ASP1/2 and P3, probes ASP 1/2 and probe P3 were found to simultaneously anneal to the DNA target such that the 5′ end of probe P3 is juxtaposed to the 3′ end of probes ASP1/2.

A typical reaction in a 20 microliter volume included 50-100 ng of genomic DNA, 5000 microspheres each of set 1 and set 2 with probes ASP1 and ASP2 bound to them, respectively, 500 nM probe P3, 2 microliters 10× Taq Ligase buffer (New England Biolabs), 500 nM of each primer S1 and AS 1, 100 micromolar each dNTP, 1.25 Units AmpliTaq Gold DNA polymerase (Perkin-Elmer) and 10 Units Taq Ligase (New England Biolabs). The various components were mixed in a single tube and subjected to the following profile in a thermal cycler: 1) 95 C. for 10 min; 2) 94 C. for 15 sec; 3) 72 C. for 30 sec; (repeat step 2 for 20 cycles); 4) 94 C. for 15 sec; 5) 55 C. for 1 min; (repeat step 4 for 30 cycles); and incubate at 4 C. until analysis. Step 1 denatured the genomic DNA and also activated the thermostable DNA polymerase (depending on the type of DNA polymerase). In Steps 2 and 3, the primers S1 and AS1 initiate PCR amplification by binding to specific regions of the genomic DNA, and produce many identical copies of a specific product. In Steps 4 and 5, probes ASP1/2 and P3 anneal to the PCR amplified DNA product and a covalent bond is formed between the 3′ end of ASP1/2 and the 5′ end of P3 enabled by the thermostable ligase enzyme. Depending on the genotype of the DNA target, one of 3 events can occur. If the target is homozygous for the base complementary to the 3′ end of ASP1, ASP1 will be joined to P3. If the target is homozygous for the base complementary to 3′ end of ASP2, ASP2 will be joined to P3. If the target is heterozygous, both ASP1 and ASP2 will be joined to P3.

Incubating the microspheres from Step 6 with streptavidin conjugated to phycoerythrin (SAPE) and analysis using a flow analyzer (Luminex100) allowed the determination of the genotype of the unknown DNA target. Oligonucleotide probe P3 had a biotin molecule at its 3′ end, such that it formed a complex with SAPE and therefore, all P3 molecules including the ones that are joined to ASP1/2 on the microsphere surface become fluorescent. The analyzer distinguished the spectral signatures associated with each microsphere set and simultaneously quantified the fluorescent reporter signal on the surface of the microspheres. Detection of a fluorescent reporter signal associated with either microsphere set 1 or set 2 alone, indicated that the DNA target was homozygous for the base complementary to 3′ end of ASP1 or ASP2, respectively. Detection of a fluorescent signal associated with both microsphere sets indicated that the DNA target was heterozygous.

The use of spectrally addressable microspheres and an appropriate flow analyzer provides for an enhanced throughput multiplex PCR/OLA assay. The spectrally addressable microspheres (LabMAP™, Luminex Corp) currently consists of 100 spectrally distinguishable microsphere sets. Also, the flow analyzer used (Luminex100 analyzer), has the capability of sampling all 100 subsets of spectrally addressable microspheres simultaneously, and can provide fluorescent reporter intensity values without the need for any additional sample processing. These properties significantly enhance the combined PCR/OLA assay's potential for performing multiplexed assays.

Again, however, it is recognized that other micosphere systems which use different fluorochromes, concentrations of fluorochromes, or other characteristics (e.g. bead size) than the ones described herein may also be used to detect the ligated products without departing from the scope of the invention

### 8. EXAMPLE 3

In this example, Embodiment 2 of a microsphere-based OLA as shown in FIG. 1 was applied in a multiplex, microsphere-based OLA to 5 of the cystic fibrosis transmembrane conductance (CFTR) mutations, including the 3 bp deletion mutation AF508. This assay also utilized the LabMAP™ system. Dunbar, et al., Clin. Chem. 46, 1498-1500 (2000). This example demonstrates the effectiveness of multiplexed OLA-based genotyping assays by coupling 10 oligonucleotide probes to distinct sets of microspheres. The free end of these oligonucleotide probes comprised of sequences corresponding to the wild type (WT) and mutant (MUT) alleles for the five mutations.

A target sample was prepared using eight genomic DNA samples previously characterized for the presence of CFTR gene mutations. The 5 loci containing the mutations to be tested were PCR amplified and the five amplicons for each sample were pooled in equimolar amounts and used as template in a multiplexed microsphere-based genotyping reaction. Other components of the OLA reaction included the 10 bound probes on microspheres, five marker-specific biotinylated free probes, reaction buffer and Taq Ligase. The target samples included homozygotes, heterozygotes mutations and compound heterozygotes, and the results of the assay are presented in Table 1.

In Table 1, the numerical results of these assays are presented as net median fluorescence intensity (MFI) values. WT and MUT indicate oligonucleotide probes for wild-type and mutant genotypes with the appropriate mutation on the 3′ end of the probe that is coupled to the microsphere. As in Example 1, the same genotyping algorithm was empirically derived, and all samples were typed correctly, which demonstrates the robustness and relative ease of developing a multiplexed OLA-based genotyping assays using spectrally addressable microspheres such as the LabMAP™ platform.

TABLE 1

| | | | | S621 + 1 G > | | | | | | | |
|---|---|---|---|---|---|---|---|---|---|---|---|
| | | ΔF508 | | T | | G542X | | N1303K | | W1282X | |
| Sample^{a} | Genotype | WT^{b,c} | MUT | WT | MUT | WT | MUT | WT | MUT | WT | MUT |
| Blank | — | 49 | 30 | 11.5 | 11 | 13 | 20 | 9 | 17.5 | 28 | 16 |
| NA12961 | V520F/Nor | 243.5 | 65.5 | 180 | 20 | 133 | 16 | 481 | 42 | 262 | 20.5 |
| NA04540 | ΔF508/ΔF508 | 65 | 243 | 235 | 20 | 177.5 | 13 | 547 | 42 | 322.5 | 25 |
| NA11496 | G542X/G542X | 305 | 80 | 171 | 18 | 17 | 85 | 468 | 36 | 254 | 15.5 |
| NA11497 | G542X/Nor | 304.5 | 77.5 | 261 | 17 | 182 | 100.5 | 710 | 57 | 404 | 30 |
| NA11274 | G551D/ΔF508 | 253 | 185.5 | 294 | 25 | 294 | 17 | 780 | 56 | 429.5 | 30 |
| NA11723 | W1282X/Nor | 303 | 79 | 302 | 19 | 298 | 17 | 817 | 71 | 299 | 160 |
| NA11472 | N1303K/ | 303 | 80 | 256 | 17 | 218.5 | 14 | 542 | 529 | 348 | 21.5 |
| | G1349D | | | | | | | | | | |
| NA11281 | 621 + 1 G > T/ | 233 | 181.5 | 91 | 74 | 176 | 18 | 552.5 | 45.5 | 297 | 22 |
| | ΔF508 | | | | | | | | | | |

Also, the overall cost per marker was minimized in some of the above assays, by performing OLA genotyping in a 5 microliter volume using as few as 1000 microspheres/probe and proportionally reducing the concentrations of all other reagents. Since the input amount of DNA template was small, the PCR reactions could also be performed in a minimal volume, for a total cost of under $0.05 per amplification. In doing so, the overall cost of OLA-based genotyping using encoded microspheres could be accomplished for under $0.10 per marker.

8.1 PCR Amplification of Target Samples

DNA samples of the various CFTR mutations were obtained from Coriell Cell Repositories (Camden, N.J.). The DNA samples were diluted to a concentration of 100 ng/μl and used directly for PCR amplifications.

PCR amplifications of the 5 fragments from CFTR gene harboring the mutations shown in Table 1 were performed in 50 μl reaction volumes using 1× PCR buffer (Qiagen, Valencia, Calif.), 1.5 mM MgCl₂, 200 μM of each dNTP, 20 pmol of each primer, 100 ng of template DNA and 2.5 U of HotStar Taq DNA polymerase (Qiagen, Valencia, Calif.). Sequences of the primers for each fragment are as follows:

| | |
|---|---|
| Exon 10: | 5′-TCTGTTCTCAGTTTTCCTGG-3′ and 5′-TTGGCATGCTTTGATGACGC-3′ |
| Exon 11: | 5′-TAGGACATCTCCAAGTTTGC-3′ and 5′-CAATAATTAGTTATTCACCTTGC-3′ |
| Exon 20: | 5′-GAGACTACTGAACACTGAAG-3′ and 5′-TTCTGGCTAAGTCCTTTTGC-3′ |
| Exon 21: | 5′-TGCTATAGAAAGTATTTATTTTTTCTGG-3′ and 5′-AGCCTTACCTCATCTGCAAC-3′ |
| Intron 4: | 5′-CTTCATCACATTGGAATGCAG-3′ and 5′-ACTTGTACCAGCTCACTACC-3′. |

Amplification reactions were incubated at 95 C. for 15 min to activate the enzyme followed by 35 cycles of denaturation at 94 C. for 30 sec, annealing at 50 C. for 1 min and extension at 72 C. for 1 min. A final extension step was performed at 72 C. for 7 min. PCR amplification for the 242 bp fragment from the HLA DQA1 was performed using primers 5′-

5′-ATGGTGTAAACTTGTACCAG-3′ and 5′-TTGGTAGCAGCGGTAGAGTTG-3′.

Amplification conditions were identical as above except the annealing temperature was 55 C.

8.2 Free and Bound Oligonucleotide Probes

All bound oligonucleotide were synthesized by Operon Inc. (Alameda, Calif.) with a 5′ UniLink™ (Clontech, Palo Alto, Calif.) amino modification. Free probes for the OLA reaction were biotinylated at the 3′ end and had a phosphate group at the 5′ end to facilitate the ligation reaction. Amino-modified bound probes were covalently coupled to 5.5 μM carboxylated latex microspheres via a one-step chemistry as described in Dunbar et al., Clin. Chem. 49: 1498-1500 (2000). Coupled microspheres were stored in 10 mM Tris-EDTA, pH 8.0 solution at a concentration of 50,000 microspheres per microliter. The sequences of the bound and free probes corresponding to the CFTR mutations are as follows:

N1303K WT-5′UnilinkTTTTTTCTGGAACATTTAGAAAAAAC-3′
N1303K MUT-5′UnilinkTTTTTTCTGGAACATTTAGAAAAAAG-3′
N1303K Free-5′pTTGGATCCCTATGAACAGTG-3′Biotin
ΔF508 WT-5′UnilinkGGCACCATTAAAGAAAATATCATCT-3′
ΔF508 MUT-5′UnilinkGGCACCATTAAAGAAAATATCA-3′
ΔF508 Free-5′pTTGGTGTTTCCTATGATGAAT-3′Biotin
W1282X WT-5′UnilinkCAATAACTTTGCAACAGTGG-3′
W1282X MUT-5′UnilinkCAATAACTTTGCAACAGTGA-3′
W1282X WT-5′pAGGAAAGCCTTTGGAG-3′Biotin
G542X WT-5′UnilinkAGAGAAAGACAATATAGTTCTTG-3′
G542X MUT-5′UnilinkAGAGAAAGACAATATAGTTCTTT-3′
G542X Free-5′pGAGAAGGTGGAATCACA-3′Biotin
621 + 1G > T WT-5′UnilinkATGTTTAGTTTGATTTATAAGAAGG-3′
621 + 1G > T MUT-5′UnilinkATGTTTAGTTTGATTTATAAGAAGT-3′
621 + 1G > T Free-5′pTAATACTTCCTTGCACAGGCC-3′Biotin

8.3 Oligonucleotide Ligation Assay.

The components of the typical OLA reaction mixture used are as follows: 5,000 microspheres of each bound probe (10,000 microspheres total in case of Embodiment 2); 5-20 ng (0.5-2 nM) of PCR amplified DNA as template; 50 nM free probe; 1× reaction buffer; and 2.5-10 units Taq Ligase (New England Biolabs, Beverly, Mass.). All reagents are present in a total reaction volume of 20 μl and are subjected to the following profile in a thermal cycler: 92° C. for 4 min; 55° C. for 3 min; and 50 cycles of 92° C. for 15 sec; and 55° C. for 30 sec. Completed OLA reactions are purified via filtration or centrifugation to eliminate excess free probes. The microspheres are then incubated with a solution containing 100 ng of streptavidin conjugated to phycoerythrin (SAPE, Molecular probes, Eugene, OR) in a volume of 100 μl for 10 min and analyzed using a Luminex100 analyzer.

8.4 Analysis using Luminex100 analyzer and Genotype Determination

The results of these assays were obtained, reported and analysed as described above in Example 1, and are summarized Table 1, above.

Although the invention has been described and illustrated in detail, it is to be clearly understood that the same is by way of illustration and example only and is not to be taken by way of limitation, the spirit and scope of this invention being limited only by the terms of the appended claims.

What is claimed is:

1. A nucleic acid ligation assay comprising:
contacting a sample suspected of containing one or more target nucleic acid sequences with one or more subsets of free probes and one or more subsets of spectrally-addressable bound probes;
allowing the one or more subsets of free probes and one or more subsets of spectrally-addressable bound probes to hybridize to the one or more target nucleic acid sequences, if present;
ligating the hybridized free probes with the hybridized spectrally-addressable bound probe, wherein a free probe hybridized to a target nucleic acid sequence is ligated with a spectrally-addressable bound probe hybridized to the same target nucleic acid sequence, to provide spectrally-addressable ligated products; and at least one of
detecting the presence of the spectrally addressable ligated products or analyzing the nucleic acid sequence of the spectrally-addressable ligated products.

2. An assay according to claim 1, wherein the sample is suspected of containing two or more target nucleic acid sequences, the sample is contacted with two or more subsets of spectrally-addressable bound probes, wherein one subset of bound probes is distinguishable from other subsets of bound probes at least based on the nucleotide sequence at the free end of the probes.

3. An assay according to claim 1, wherein:
the sample is suspected of containing one or more first and one or more second target nucleic acid sequences, the one or more first target nucleic acid sequences have at least a first portion and a second portion and the one or more second target nucleic acid sequences have at least a first portion and a second portion, and wherein the first portion of the one or more first target nucleic acid sequences is distinguishable from the first portion of the one or more second target nucleic acid sequences but the second portion of the one or more first target nucleic acid sequences is substantially identical to the second portion of the one or more second target nucleic acid sequences; and
the sample is contacted with two subsets of spectrally-addressable bound probes and one subset of free probes, wherein the first subset of spectrally-addressable bound probes is specific for the first portion of the one or more first target nucleic acid sequences and the second set of spectrally-addressable bound probes is specific for the first portion of the one or more second target nucleic acid sequences and the free probes have substantially identical nucleotide sequences specific for the second portion of the one or more first and second target nucleic acid sequences.

4. An assay according to claim 1, wherein the sample is suspected of containing two or more target nucleic acid sequences, the sample is contacted with two or more subsets of free probes, wherein one subset of free probes is distinguishable from other subsets of free probes at least based on the nucleotide sequence at the free end of the probes.

5. An assay according to claim 1, wherein:
the sample is suspected of containing one or more first and one or more second target nucleic acid sequences, the one or more first target nucleic acid sequences have at least a first portion and a second portion and the one or more second target nucleic acid sequences have at least a first portion and a second portion, and wherein the first portion of the one or more first target nucleic acid sequences is distinguishable from the first portion of the one or more second target nucleic acid sequences but the second portion of the one or more first target nucleic acid sequences is substantially identical to the second portion of the one or more second target nucleic acid sequences; and
the sample is contacted with two subsets of free probes and one subset of spectrally-addressable bound probes, wherein the first subset of free probes is specific for the first portion of the one or more first target nucleic acid sequences and the second set of spectrally-addressable bound probes is specific for the first portion of the one or more second target nucleic acid sequences and the one subset of spectrally-addressable bound probes have substantially identical nucleotide sequences specific for the second portion of the one or more first and second target nucleic acid sequences.

6. An assay according to claim 5, wherein the assay is performed in a first and a second reaction vessel, a portion of the sample is contacted with the first subset of free probes in the first reaction vessel and a portion of the sample is contacted with the second subset of free probes in the second reaction vessel.

7. An assay according to claim 1, further comprising using a thermostable ligase for ligating the probes.

8. An assay according to claim 1, wherein a substantially same amount of at least one fluorescent dye is incorporated into each bound probe in a subset, and one subset of spectrally-addressable bound probes is distinguishable from other subsets of spectrally-addressable bound probes based at least on the relative amount of the at least one fluorescent dye incorporated into the spectrally-addressable bound probe of the subset.

13. An assay according to claim 1, wherein the assay further comprises contacting the sample with polymerase chain reaction components and amplifying the target nucleic acid molecule.

14. A microsphere-based oligonucleotide ligation assay comprising:
(a) contacting a sample, which is suspected of containing target nucleic acid molecules having a certain nucleotide sequence, with a mixture comprising at least one set of free probes and at least one subset of microspheres to which are coupled bound probes, wherein (i) the free probes of a given set comprise a detectable label at one of their ends and an oligonucleotide having a predetermined nucleotide sequence that is complementary to at least a portion of the target nucleic acid molecules, (ii) the bound probes of a given subset of microspheres comprise a modifier moiety, which is used for coupling a bound probe to a microsphere, at one of their ends and an oligonucleotide having a predetermined nucleotide sequence that is complementary to at least another portion of the target nucleic acid molecules, and (iii) the microspheres of a given subset having a unique spectral address, which allows one to distinguish the microspheres of a given subset from those of another;
(b) allowing the free probes and the bound probes to hybridize to the target nucleic acid molecules,
(c) ligating one of the ends of the free probes with one of the ends of the bound probes to provide microsphere-bound ligated products; and
(d) detecting the presence of microsphere-bound ligated products.

15. The assay of claim 14 in which the free probes and the bound probes are allowed to hybridize to different portions of the target nucleic acid molecules.

16. The assay of claim 15 in which the different portions of the target nucleic acid molecules do not overlap.

17. The assay of claim 14 in which the free probes further comprise a phosphate at the other of their ends.

18. The assay of claim 14 in which the bound probes further comprise a phosphate at the other of their ends.

19. The assay of claim 14 in which the mixture comprises at least two subsets of microspheres, the bound probes coupled to the microspheres of one subset being different from those coupled to the microspheres of the other subset.

20. The assay of claim 19 in which the bound probes differ in that the nucleotide found at one end of one subset differs from that found at the corresponding end of the other subset, wherein the nucleotide sequences comprising the at least two subsets of bound probes are otherwise substantially identical.

21. The assay of claim 20 in which the mixture comprises free probes having substantially identical nucleotide sequences.

22. The assay of claim 19 in which the bound probes differ in the identity of one or more nucleotides at one or more positions of the predetermined nucleotide sequence.

23. The assay of claim 19 in which the bound probes differ due to one or more substitutions, insertions, deletions, or combinations thereof, at one or more positions of the predetermined nucleotide sequence.

24. The assay of claim 15 in which the mixture comprises at least two sets of free probes, the nucleotide and the detectable label found at opposite ends of one set differing from the nucleotide and the detectable label found in the corresponding ends of the other set, wherein the nucleotide sequences comprising the at least two sets of free probes are otherwise substantially identical.

25. The assay of claim 24 in which the mixture comprises bound probes having substantially identical nucleotide sequences.

26. The assay of claim 14 in which the oligonucleotides of the at least one set of free probes and at least one subset of microspheres have 5′ and 3′ ends, and wherein the free probes of a given set include a phosphate at their 5′ ends and a detectable label at their 3′ ends; and the modifier moiety is an amine which couples the 5′ end of the oligonucleotide of the bound probe to a carboxylic acid group on the microsphere.

27. The assay of claim 26 in which the mixture comprises at least two subsets of microspheres, the bound probes coupled to the microspheres of one subset being different from the bound probes coupled to the microspheres of the other subset in that a portion of the oligonucleotide at the 3′ end of one subset differs from the a portion of the oligonucleotide at the 3′ end of the other subset, wherein the nucleotide sequences comprising the at least two subsets of bound probes are otherwise substantially identical.

28. The assay of claim 26 in which the mixture comprises at least two sets of free probes, the portion of the oligonucleotide found at the 5′ end of one set differing from the portion of the oligonucleotide at the 5′ends of the other set, wherein the nucleotide sequences comprising the at least two sets of free probes are otherwise substantially identical.

29. The assay of claim 26 which is carried out in substantially the same reaction vessel.

30. The assay of claim 26 which is carried out in separate reaction vessels, at least one for each set of free probes.

31. The assay of claim 27 in which the microspheres of one subset can be distinguished from the microspheres of the other subset in that the microspheres of the one subset harbor at least one fluorescent dye at a concentration which differs from the concentration of the at least one fluorescent dye harbored by the microspheres of the other subset.

33. The assay of claim 27 in which the spectrally addressable microspheres of one subset can be distinguished from the spectrally addressable microspheres of another subset by the relative amounts of at least two fluorescent dyes harbored by the spectrally addressable microspheres.

34. The assay of claim 26, wherein the mixture further comprises polymerase chain reaction components, and wherein the assay further comprises the step of amplifying a portion of the target nucleic acid molecule.

35. A kit for performing a microsphere-based oligonucleotide ligation assay comprising bound probes coupled to spectrally addressable microspheres and free probes bearing a detectable label.

36. The kit of claim 35 in which at least two subsets of microspheres are present.

37. The kit of claim 36 which further comprises a thermostable ligase.

38. The kit of claim 36 which further comprises one or more reagents for effecting nucleic acid amplification.

39. The kit of claim 38 which further comprises one or more reaction buffers.

40. The assay of claim 14 in which the modifier moiety comprises an amine modifier moiety.

41. The assay of claim 14 in which the modifier moiety comprises a primary amine group for coupling the bound probe to a carboxylic acid group of the microsphere.
